(19) United States
(12) Patent Application Publication
(10) Pub. No.: US 2004/0146460 A1
(43) Pub. Date: Jul. 29, 2004

(54) Design of labels for detection with a surface-seletive nonlinear optical technique

(76) Inventor: Joshua S Salafsky, New York, NY (US)

Correspondence Address: JONES DAY, 222 EAST 41ST ST, NEW YORK, NY, 10017, US

(21) Appl. No.: 10/467,098

(22) PCT Filed: Jul. 17, 2001
(86) PCT No.: PCT/US01/22412

(51) Int. Cl. G01N 33/00; A61K 49/00

(52) U.S. Cl. 424/9.6; 252/582; 435/7.1

(57) ABSTRACT

Second harmonic, sum and difference frequency generation can be used to detect a variety of processes, which are otherwise undetectable, using nonlinear-active labels. The labels must have as high a hyperpolarizability as possible. Several designs for large hyperpolarizability second-harmonic active labels are described herein. These labels can be attached to any target molecule or particle, resulting in more highly nonlinear-optically active targets.

### BACKGROUND OF THE INVENTION

The prior art shows that second harmonic-active moieties, when coupled to a protein as a label, can render the protein detectable at an interface by second harmonic generation. In this case, the adsorption of a labeled protein at an air-water interface was monitored to measure the adsorption curve and surface density of protein. The second harmonic-active label was an oxazole dye derivative which could be covalently coupled to surface amines or sulfhydryls on the protein's surface. Single-molecule labels may not provide enough scattering cross-section and the target objects—such as cells, proteins, viruses or nucleic acids—may have only one or several sites available for labeling and these are typically at random orientations to each other, further reducing the net cross-section. Because the second harmonic labels used to date may not be sufficient to monitor many processes of interest, designs of labels with significantly higher hyperpolarizabilities (eg., second harmonic cross-sections) are needed.

Relevant portions of cited references are incorporated by reference herein.

### DESCRIPTION OF THE INVENTION

The present invention offers a number of designs of second-harmonic active labels with high hyperpolarizabilities. Designs are proposed which involve the use of non-centrosymmetric metallic particles, oxazole dyes, linear chains of nonlinear-active dyes and solid scaffolds on which to build nonlinear active moieties, nanocrystals or nanoparticles. These high-cross-section (hyperpolarizability) labels may then be used to label viruses, cells, proteins, nucleic acids or other particles, especially biological particles (“bioparticles”).

One means of determining whether a particular molecule or particle is a candidate for use as a nonlinear-active label is by studying it using second harmonic generation at an air-water interface. For instance, in the case of particles, if the particles assemble at the air-water interface in a manner which gives a net orientation of the particles (on a length scale of the coherence length) the layer of particles will generate second harmonic light. Another means of doing this is by measuring a sample of a suspension of the particles and detecting the hyper-rayleigh scattering. Yet another means is by EFISH (Electric-field induced second harmonic generation). EFISH can be used to determine if a candidate molecule or particle is nonlinearly active. Electric field induced second harmonic (EFISH) is well known in the field of nonlinear optics. This is a third order nonlinear optical effect, with the polarization source written as: P⁽²⁾(ω₃)=χ⁽²⁾(−ω₃)=χ(²⁾(−ω₃;ω₁,ω₂): E^{ω1} E^{ω2}. The effect can be used to measure the hyperpolarizabilty of molecules in solution by using a dc field to induce alignment in the medium, and allowing SHG to be observed. This type of measurement does not require that the particle themselves be ordered at an interface, but does require that the particles be nonlinear active.

In one important aspect of the invention, the use of linkers which couple the labels to their targets can be made long enough so that the orientation of the targets at the interface does not significantly affect the orientation of the label. Because the intensity of the nonlinear light generated will depend on the net orientation of the labels at the interface—and the orientation of the targets at an interface can be difficult to control (i.e., the targets may even be randomly oriented at the interface)—the use of linkers can separate the labels sufficiently from the targets so that the orientation of the targets does not necessarily determine the orientation of the labels, resulting in a net orientation of labels. In cases where this is less important, for example with integral membrane proteins in supported lipid bilayers on glass—where the orientation of the membrane protein presented to the targets is generally uniform—this aspect of the linkers can be less important. Nevertheless, in many cases, linkers are necessary in order to couple the label to the targets.

Certain terms used herein are intended to have the following general definitions:

1. Complementary: Refers to the topological and chemical compatibility of interacting surfaces between two biological components, such as with a ligand molecule and its receptor (also referred to in the prior art as: ‘molecular recognition’). Thus, the receptor and its ligand can be described as complementary, and, furthermore, the contacts' surface characteristics are complementary to each other.

2. Biological (Components): These may include any naturally occurring or modified particles or molecules found in biology, or those molecules and particles which are employed in a biological study. Examples of these include, but are not limited to, a biological cell, protein, nucleic acids, antibodies, receptors, peptides, small molecules, oligonucleotides, carbohydrates, lipids, liposomes, polynucleotides and others such as drugs, toxins and genetically engineered protein or peptide.

3. Ligand: A ligand is a molecule that is recognized by a particular receptor. Examples of ligands that can be studied by this invention include, but are not restricted to, antagonists or agonists for cell membrane receptors, toxins and venoms, viral epitopes, hormones, hormone receptors, peptides, enzymes, enzyme substrates, cofactors, drugs (e.g. opiates, steroides, etc.), lectins, sugars, oligonucleotides, nucleic acids, oligosaccharides, proteins, and monoclonal antibodies.

4. Receptor: A molecule that has an affinity for a given ligand. Receptors may be naturally occurring or man-made molecules. Also, they can be used in an unaltered state or as aggregates with other species. Receptors may be attached, covalently or noncovalently, to a binding partner, either directly or via a specific binding substance. Examples of receptors which can be employed by this invention include, but are not limited to, antibodies, cell membrane receptors, monoclonal antibodies and antisera reactive with specific antigenic deteminants (such as on viruses, cells or other materials), drugs, polynucleotides, nucleic acids, peptides, cofactors, lectins, sugars, polysaccharides, cells, cellular membranes and organelles. Receptors are occasionally referred to in the art as anti-ligand. As the term receptors is used herein, no difference in meaning is intended. A “Ligand Receptor Pair” is formed when two macromolecules have combined through molecular recognition to form a complex.

Other examples of receptors which can be investigated by this invention include but are not restricted to:

a) Microorganism receptors: Determination of ligands which bind to receptors, such as specific transport proteins or enzymes essential to survival of microorganisms,

is useful in developing a new class of antibiotics. Of particular value would be antibiotics against opportunistic fungi, protozoa, and those bacteria resistant to the

antibiotics in current use.

b) Enzymes: For instance, one type of receptor is the binding site of enzymes such as the enzymes responsible for cleaving neurotransmitters; determination of ligands

which bind to certain receptors to modulate the action of the enzymes which cleave the different neurotransmitters is useful in the development of drugs which can be

used in the treatment of disorders of neurotransmission.

c) Antibodies: For instance, the invention may be useful in investigating the ligand-binding site on the antibody molecule which combines with the epitope of an

antigen of interest; determining a sequence that mimics an antigenic epitope may lead to the development of vaccines of which the immunogen is based on one or more of such sequences or lead to the development of related diagnostic agents or compounds useful in therapeutic treatments such as for autoimmune diseases (e.g., by blocking the binding of the “self” antibodies).

d) Nucleic Acids: Sequences of nucleic acids may be synthesized to establish DNA or RNA binding sequences.

e) Catalytic Polypeptides: Polymers, preferably polypeptides, which are capable of promoting a chemical reaction involving the conversion of one or more reactants

to one or more products. Such polypeptides generally include a binding site specific for at least one reactant or reaction intermediate and an active functionality

proximate to the binding site, which functionality is capable of chemically modifying the bound reactant. Catalytic polypeptides are described in, for example, U.S. Pat. No. 5,215,899, which is incorporated herein by reference for all purposes.

f) Hormone receptors: Examples of hormone receptors include, e.g., the receptors for insulin and growth hormone. Determination of the ligands which bind with high

affinity to a receptor is useful in the development of, for example, an oral replacement of the daily injections which diabetics must take to relieve the symptoms of

diabetes, and in the other case, a replacement for the scarce human growth hormone which can only be obtained from cadavers or by recombinant DNA

technology. Other examples are the vasoconstrictive hormone receptors; determination of those ligands which bind to a receptor may lead to the development of

drugs to control blood pressure.

g) Opiate receptors: Determination of ligands which bind to the opiate receptors in the brain is useful in the development of less-addictive replacements for morphine

and related drugs.

h) Ion channel proteins or receptors, or cells containing ion channel receptors.

5. Surface-selective: Refers to a non-linear optical technique such as second harmonic generation or sum/difference frequency generation in which, by symmetry, only a non-centrosymmetric surface (comprising array, substrate, solution, biological components, etc.), is capable of generating non-linear light.

6. Array: Refers to a substrate or solid support on which is fabricated one type, or a plurality of types, of biological components in one or a plurality of known locations. This includes, but is not limited to, two-dimensional microarrays and other patterned samples. Other terms in the prior art which are often used interchangeably for ‘array’ include: gene chip, gene array, biochip, DNA chip, protein chip and microarray.

7. Label: Refers to a nonlinear-active moiety, particle or molecule which can be attached (covalently or non-covalently) to a molecule, particle or phase (e.g., lipid bilayer) in order to render the latter more nonlinear optical active. The labels are pre-attached to the molecules or particles and unbound or unreacted labels separated from the labeled entities before a measurement is made.

8. Linker: A molecule which serves to chemically link (usually via covalent bonds) two different objects together. Herein a linker can be used to couple targets to non-linear active particles or moieties, targets to nonlinear-active derivatized particles, surface layers to targets, surface layers to nonlinear-active particle or moieties, etc. A linker may be a homobifunctional or heterobifunctional cross-linker molecule, a biotin-streptavidin couple wherein the biotin is attached to one of the two objects and the streptavidin to the other, etc. Many linkers are available commercially, for example from Pierce Chemical Inc., Sigma-Aldrich, Fluka, etc. In some prior art, the term ‘tether’, ‘spacer’ or ‘cross-linker’ is also used with the same meaning.

9. Elements: When used with ‘array’ or ‘microarray’, the meaning is a specific location among the plurality of locations on the array surface. Each element is a discrete region of finite area formed on the surface of a solid support or substrate.

10. Nonlinear: Refers herein to those optical techniques capable of transforming the frequency of an incident light beam (called the fundamental). The nonlinear beams are the higher order frequency beams which result from such a transformation, e.g. second harmonic, etc. In second harmonic, sum frequency or difference frequency generation, the nonlinear beams are generated coherently. In second harmonic generation (SHG), two photons of the fundamental beam are virtually scattered by the interface to produce one photon of the second harmonic. Also referred to herein as nonlinear optical or surface-selective nonlinear (optical) or by various combinations thereof.

11. Target: Refers herein to a particle or molecule to be labeled with a nonlinear-active moiety, in order to render said particle or molecule for study by a nonlinear-active technique at an interface of interest. Biological targets, for example, may include the following: a protein, oligosaccharide, peptide, nucleic acid, liposome, small molecule, oligonucleotide, liposome, or biological cell, liposome, receptor, antibody, antigen, peptide, receptor, drug, enzyme, ligand, carbohydrate.

12. Attached: Refers herein to biological components which are either prepared or engineered in-vitro to be attached to some surface, via covalent or non-covalent means, including for example the use of linker molecules; or are naturally part of the surface such as in the example of membrane receptors embedded in cell membranes, liposomes, tissues, organs (in-vitro or in-vivo) or supported lipid bilayer membranes.

13. Centrosymmetric: A molecule or material phase is centrosymmetric if there exists a point in space (the ‘center’) through which an inversion (x,y,z)→(−x,−y,−z) of all atoms is performed that leaves the molecule or material unchanged. A non-centrosymmetric molecule or material lacks this center of inversion. For example, if the molecule is of uniform composition and spherical or cubic in shape, it is centrosymmetric.

14. Nucleic Acid Analog: A non-natural nucleic acid which can function as a natural nucleic acid in some way. For example, a Peptide Nucleic Acid (PNA) is a non-natural nucleic acid because it has a peptide-like backbone rather than the phosphate background of natural nucleic acids. The PNAs can hybridize to natural nucleic acids via base-pair interactions. Another example of a Nucleic acid analog can be one in which the base pairs are non-natural in some way.

15. Binding Affinity or Affinity: The specific physico-chemical interactions between binding partners, such as a probe and target, which lead to a binding complex (affinity) between them. The binding reaction is characterized by an equilibrium constant which is a measure of the energetic strength of binding between the partners. Specificity in a binding reaction implies that probe-target binding only occurs appreciably with specific binding partners—not any at random. For example, the protein Immunoglobulin G (IgG) has a specific binding affinity for protein G and not for other proteins. In some prior art, the term ‘molecular recognition’ is used to describe the binding affinity between components.

16. Electrically Charged or Electric Charge: Defined herein as net electric charge on a particle or molecule, which confers a mobility (velocity) of said particle or molecule in an electric field. The net charge could be part of a molecular moiety such as phosphate group on nucleic acid backbones, side-chains of amino acid residues in proteins, lipid head groups in membrane lipids or cellular membranes, etc. The charge can be positive or negative and would determine the direction of mobility of the particle or molecule if said particle or molecule is placed in an electric field of a given orientation (direction of positive to negative electric potential). The charge can be non-integer multiples of the fundamental unit of charge (q≈1.6×10⁻¹⁹ C) or a fraction of the fundamental unit of charge—so-called ‘partial charges’, well known to those skilled in the art.

17. Dipolar: Defined herein as possessing an electric dipole or ‘dipole moment’ (no net charge) on a particle or molecule, which takes the standard definition known to one skilled in the art: the sum of all vectors μ=Q.R where Q is the amount of charge (positive or negative) at a particular spatial location (x,y,z in Cartesian coordinates) in the particle or molecule and R is the vector which points from an origin of reference (x,y,z) to the net charge Q. If the sum of these vectors results in a vector with a non-zero trace (sum of x,y,z components of the resultant vector), the particle or molecule possesses a dipole moment and is electrically dipolar.

18. Electrically Neutral: Defined herein as zero net (sum of positive and negative) electric charge on a particle or molecule, which would result in no appreciable mobility (velocity) of said particle or molecule in an electric field.

19. Hyperpolarizability or Nonlinear Susceptibility: The properties of a molecule, particle, interface or phase which allows for generation of the nonlinear light. Typical equations describing the nonlinear interaction for second harmonic generation are: α⁽²⁾(2ω)=β:E(ω).E(ω) or P⁽²⁾(2ω)=χ⁽²⁾:E(ω)E(ω) where α and P are, respectively, the induced molecular and macroscopic dipoles oscillating at frequency 2ω, β and χ⁽²⁾ are, respectively, the hyperpolarizability and second-harmonic (nonlinear) susceptibility tensors, and E(ω) is the electric field component of the incident radiation oscillating at frequency ω. The macroscopic nonlinear susceptibility χ⁽²⁾ is related by an orientational average of the microscopic β hyperpolarizability. For sum or difference frequency generation, the driving electric fields (fundamentals) oscillate at different frequencies (i.e., ω₁ and ω₂ and the nonlinear radiation oscillates at the sum or difference frequency (ω¹±ω₂). The terms hyperpolarizability, second-order nonlinear polarizability and nonlinear susceptibility are sometimes used interchangeably, although the latter term generally refers to the macroscopic nonlinear-activity of a material or chemical phase or interface. The terms ‘nonlinear active’ or ‘nonlinearly active’ used herein also refer to the general property of the ability of molecules, particles, an interface or a phase, to generate nonlinear optical radiation when driven by incident radiation beam or beams.

20. Polarization: The net dipole per unit volume (or area) in a region of space. The polarization can be time-dependent or stationary. Polarization is defined as: ∫μ(R) dR where an integration of the net dipole is made over all volume elements in space dR near an interface.

21. Radiation: Refers herein to electromagnetic radiation or light, including the fundamental beams used to generate the nonlinear optical effect, or the nonlinear optical beams which are generated by the fundamental.

22. Near-field techniques: Those techniques known in the prior art to be capable of measuring or imaging optical radiation on a surface or substrate with a lateral resolution at or smaller than the diffraction-limited distance. Examples of near-field techniques (or near-field imaging) include NSOM (near-field scanning optical microscopy) whereby optical radiation (from fluorescence, second harmonic generation, etc.) is collected at a point very near the surface.

23. Detecting: Refers herein to methods by which the properties of surface-selective nonlinear optical radiation can be used to detect, measure or correlate properties of probe-target binding reactions or effects of the binding reactions.

24. Interface: For the purpose of this invention, the interface can be defined as that region which generates a nonlinear optical signal.

25. Surface layer: Refers herein to a chemical layer which functionally derivatizes the surface of a solid support. For instance, the surface chemical groups can be changed by the derivatization layer according to the particular chemical functionality of the derivatizing agent. In the case of solid objects used as ‘scaffolds’ for creating power nonlinear-active labels (see below), the solid surface can be derivatized to produce a different chemical functionality which can be presented to nonlinear active moieties or particles, or to targets. For instance, a silica bead with negatively charged silanol groups on its surface can be converted to an amine-reactive, amine-containing, etc. surface via organosilane reagents.

26. Conjugated: Refers herein to the state in which one particle, moiety or molecule is chemically bonded, covalently or non-covalently linked or otherwise attached to a second particle moiety or molecule. The second particle, moiety or molecule is often a target, i.e. a species of interest which must be labeled for detection by a nonlinear optical technique.

Organic Molecules

It was demonstrated that an oxazole dye 4-[5-methoxyphenyl)-2-oxazolyl]pyridinium methanesulfonate (also known as 4PyMPO-MeMs) is strongly second harmonic-active and chemically stable at neutral pH (Salafsky and Eisenthal, Chemical Physics Letters). Furthermore, the Stokes shift of the fluorescence which results from two-photon absorption is large so that the second harmonic beam can readily be separated from the fluorescence. Other dyes in this family have similar properties, including but not limited to (J. H. Hall, 1992):

5-(4-methoxphenyl)-2-(4-methoxyphenyl)-2-(4-pyridyl)oxazole

2-(4-methoxyphenyl)-5-(4-pyridyl)oxazole

2-(4-methoxyphenyl)-5-(4-pyridyl)oxadiazole

2-(4-methoxyphenyl)-5-(4-pyridyl)furan

2-(4-pyridyl)-4,5-dihydronapthol[1,2-d]-1,3-oxazole

5-Aryl-2-(4-pyridyl)4R-oxazole where R is a hydrogen atom, methyl group, ethyl group or other akyl group.

2-(4-pyridyl)cycloalkano[d]oxazole

2-(4-pyridyl)phenanthreno[9,10-d]-1,3-oxazole

6-Methoxy-4,4-dimethyl-2-(4-pyridyl)indeno[2,1-d]oxazole

4,5-Dihydro-7-methoxy-2-(4-pyridyl)napthol[1,2-d]-1,3-oxazole

These dyes can readily be made into labels, that is, reactive to various functional groups on targets, by using synthetic methods known to one skilled in the art. Furthermore, the following two commercially available dyes (Molecular Probes, Inc.) can be readily conjugated—without further modification—to protein amines [1] or cysteines [2].

[1] 1-(3-(succinimidyloxycarbonyl) benzyl)-4-(5-(4-methoxyphenyl) oxazol-2-yl)pyridinium bromide.

[2] 1-(2,3-epoxypropyl)-4-(5-(4-methoxyphenyl)oxazol-2-yl) pyridinium trifluoromethanesulfonate (PyMPO epoxide).

Other molecules which can, by synthetic means known to one skilled in the art, be made reactive to various functional groups on targets include members of the following families:

Merocyanines

Stilbenes

Indodicarbocyanines

Hemicyanines

Stilbazims

Azo dyes

Cyanines

Stryryl-based dyes

Methylene blue

Diaminobenzene compounds

Polyenes

Diazostilbenes

Tricyanovinyl aniline

Tricyanovinyl azo

Melamines

Phenothiazine-stilbazole

Polyimide

Sulphonyl-substituted azobenzenes

Indandione-1,3-pyidinium betaine

Fluorescein

Benzooxazole

Perylene

Polymethacrylates

Oxonol

Thiophenes

Bithiophenes

Derivatized Particle Labels

A solid microparticle or a nanoparticle of size nanometers to microns in scale including, but not limited to, a sphere (latex, polystyrene, silica, etc.) or a strip, offers a surface area which can be derivatized with a nonlinear-active moiety via chemical or electrostatic means so that the entire object has a much higher hyperpolarizability than may be obtained otherwise. For instance, nonlinear-active dyes can be assembled on silica bead surfaces via electrostatic interactions (dye is positively charged, silica surface is negatively charged) and the entire bead, if derivatized with target-reactive linkers, can then function as a nonlinear active label. If the nonlinear active moieties are assembled on the solid surface so that phase interference between moieties is small, the overall hyperpolarizability will scale nonlinearly (eg., quadratically) in their number. The solid particle can vary in shape and its size can range from nanometers to microns in scale. Linkers which allow attachment to the target object (e.g., cells, viruses, proteins, nucleic acid) can be attached to the particle surface—at low density if necessary, for example to prevent multiple attachment points of the label target to the label—using commercially available bifunctional linkers. The nonlinear active moieties will ideally all have the same orientation, or the same orientation with respect to the solid particle, for an optimal scattering cross-section.

Examples of the particles to be used include, but are not limited to, polystyrene beads and silica beads, both readily commercially available.

a. Covalent Attachment

The solid particles can be surface derivatized using a variety of chemistries available in the prior art. Nonlinear-active moieties are covalently coupled either to the solid particles or to a derivatized layer. The nonlinear-active moieties themselves can contain linkers for making the covalent attachment, if necessary.

For instance, polystyrene beads can be derivatized with dextran, lactose or amines (the latter case for example, via chloromethyl groups with ethylenediamine). Silica can be derivatized using organofunctional silanes, for example using trichlorosilanes or other functional silanes (such as methoxy, amine, or other functional groups), to produce surfaces with a variety of chemical functionalities. The surfaces of the derivatized beads can then be reacted with a nonlinear active moiety via appropriate chemistry.

b. Electrostatic Attachment

Nonlinear active moieties can also be electrostatically bound to a micron- or nanometer-sized particle surface. This has been demonstrated in the prior art with charged nonlinear active moieties using silica or polystyrene beads and malachite green or oxazole dyes. A charged nonlinear active moiety, an organic dye for example, can be oriented at a counter-charged microparticle surface, thus allowing for a net hyperpolarizability of the object when using an appropriate geometry. An example of an appropriate geometry is a microparticle sphere where the diameter is approximately the wavelength of the fundamental light, i.e. from tens of nanometers to microns so that destructive phase interference between nonlinear active moieties on opposing faces of the sphere is minimized. The hyperpolarizability of each dye at the spheres's surface, when integrated across the entire surface of the sphere of ˜wavelength of light size, is large and positive.

Preferred Embodiment:

For instance, a commercially available oxazole dye with functionality for binding to amines (1-(3-(succinimidyloxycarbonyl) benzyl)-4-(5-(4-methoxyphenyl) oxazol-2-yl)pyridinium bromide (PyMPO-SE) can be coupled to beads with amine or surface groups. Spherical silica beads (˜200 nm diameter) are derivatized with surface amines using 3-aminopropyltrimethoxysilane according to means well known in the prior art. The PyMPO dye is then reacted with the beads, covalently linking the dye to the surface of the beads. About 5-10% of the available amines can be left unreacted with the dye by varying the reaction conditions. These unreacted amines can then be covalently coupled to a heterobifunctional crosslinking agent N(4-Azidosalicylamido)Butyl 3′(2′Pyridyldithio)Propionamide (available from Pierce Chemical, Inc.). The crosslinker allows covalent attachment to amines and sulfhydryls. The sulfhydryl-functional end is available for reaction with sulfhydryl groups on the target's surface.

Preferred Embodiment:

Silica beads (˜200 nm, roughly spherical) are reacted with a low concentration of 3-aminopropyltrimethoxysilane or 3-aminooctyltrimethoxysilane so that only ˜5-10% of the surface silanols become covalently coupled to the silane agent. These amine groups are then reacted with the amine-reactive homobifunctional crosslinker Disuccinimidyl glutarate (DSG, Pierce Chemical) to create amine-reactive linkers on ˜5-10% of the bead surface. The beads are then incubated with 4-[5-methoxyphenyl)-2-oxazolyl]pyridinium methanesulfonate (also known as 4PyMPO-MeMs), a positively charged dye which binds electrostatically to the charged silanols on the surface and orients to some degree. The excess dye is removed from the beads by centrifugation. The electrostatic adsorption can be sufficiently high in some cases to immobilize the charged dye, even in the absence of a bulk concentration of it. The beads can then be attached covalently to target objects containing amines using the amine-reactive tethers. This coupling can produce target objects containing amines (eg., proteins, viruses, cells, oligonucleotides, nucleic acids, etc.) coupled to a strongly nonlinear-active label.

FIG. 1 illustrates in (A) two possibilities for an organic nonlinear-active molecule or moiety. (B) illustrates some of the various possibilities involving linkers, solid objects (‘scaffolds’) and non-linear active components, the components being molecules, particles, proteins or moieties which are nonlinear active.

LINEAR CHAINS OF NONLINEAR ACTIVE MOIETIES

Linear chains of nonlinear active moieties which are aligned in a manner to maximize the overall scattering cross-section and have functionality for attaching to target objects would be useful as nonlinear-active labels. For example, rigid nonlinear active dyes can be coupled in a head-to-tail polymeric fashion, with each monomer of dye oriented in the same direction. For example, dyes such as the oxazole can be created so that they can covalently connect to each other in a head-to-tail fashion, and with a coupling moiety at one end only for attachment to the target object. For example, as depicted in FIG. 3, by reacting a nonlinear active molecule containing functional group (X) with an excess of a nonlinear-active molecule containing both (X) and (Y) where (Y) is reactive towards (X), one could easily construct chains of monomers of the dye under conditions where (X) and (Y) are reactive. By synthetic means available to one skilled in the art, one could easily functionalize nonlinear-active molecules or moieties with (X) and (Y) groups.

DERIVATIZED NON-CENTROSYMMETRIC, METALLIC NANOCRYSTALS, NANOPARTICLES, CLUSTERS AND COLLOIDS

Prior art shows that metallic nanoparticles and clusters, ranging from about 1 nm to 25 or more microns in size, can be derivatized and conjugated to biomolecules for use in staining for electron microscopy, x-ray scattering and other applications. Prior art also shows that non-centrosymmetric metal nanoparticles can exhibit extremely high hyperpolarizabilities (3,5,9).

Another aspect of the present invention therefore is to use non-centrosymmetric metal nanocrystals or nanoparticles as labels for nonlinear optical studies. A variety of shapes and sizes of metal nanoparticles are available in the prior art. To use these particles as labels, one must derivatize them for conjugation to a target biological component or particle. Either the labels must be derivatized with linkers; or the targets must be derivatized with linkers allowing for their coupling to the labels (the labels can be derivatized if necessary). A number of embodiments employing these metal particles are described herein.

FIG. 2 depicts some of the various combinations of linkers and metallic or semiconductor particles. The particles can be both centrosymmetric or non-centrosymmetric. If centrosymmetric, they must be joined together in clusters to create a composite particle which is overall non-centrosymmetric; or they must be greater than or equal to 10% of the wavelength of the fundamental light used in the nonlinear optical technique.

Preferred Embodiment:

Non-centrosymmetric gold particles can be prepared by lithographic means (according to means found in references 2 and 5) or by synthetic methods (according to means found in reference 7). These can then be derivatized with X—R—SH where —SH is a sulfhydryl moiety, R is an alkyl chain and X is a terminal group suitable for conjugation to amines or sulfhydryls. The gold particles can be derivatized, for example, with HS—(CH₂)₁₅—COOH (obtained commercially) according to means well known in the prior art. The carboxyl groups on the derivatized particle can then be made amine-reactive by reaction with 1-ethyl-3-(3-dimethylaminopropyl)carbodiimide (EDAC, Molecular Probes, Inc.) according to protocols supplied by Molecular Probes. The particles are now amine-reactive and can be conjugated directly to protein amines, or to protein sulfhydryls via a variety of heterobifunctional crosslinkers which are commercially available (Pierce Inc., Rockford, Il.)

In an alternate embodiment, the carboxyl group of the functional alkylthiol listed above can be coupled to oligonucleotides or nucleic acids via similar synthetic means.

In an alternate embodiment, a tris(aryl) phosphine ligand bearing a single primary amine is mixed with tris(p-N-methylcarboxamidophenyl) in a ratio 1:5 to derivatize the gold particle as described in U.S. Pat. No. 5,521,289, which reference is incorporated by reference herein. The gold particles are then reacted with N methoxycarbonylmaleimide (NMCM) in DMSO, mixed and incubated at 0 degrees C. for 30 minutes. The maleimido-gold particles can be separated from unreacted NMCM on a gel filtration column. The maleimido-gold particles can then be reacted with a variety of biological moieties including amines, sulfhydryls and carboxylic acids by using cross-linking agents if necessary.

In an alternate embodiment, a tris(aryl)phosphine ligand bearing a single nonlinear active moiety (eg., oxazole dye) is reacted with the tris(aryl)phosphine ligand and tris(p-N-methylcarboxamidophenyl) as described in U.S. Pat. No. 5,521,289 to produce particles which contain nonlinear active molecules as well as the means to make them reactive with biological molecules.

In an alternate embodiment, silica beads can be used (readily available from a number of commercial sources). A bead with ˜200 nm diameter is reacted with 3-aminopropyltrimethoxysilane so that the charged silanols are functionalized to produce an amine-surface. These beads can then be reacted with a mixture of 1-(3-(succinimidyloxycarbonyl) benzyl)-4-(5-(4-methoxyphenyl) oxazol-2-yl)pyridinium bromide (YMPO-SE; Molecular Probes Inc.) and (N-[-Maleimidobutyryloxy]succinimide ester (GMBS, Pierce Chemical Inc.). This reaction produces a bead with a surface that has both a covalently attached oxazole dye and a tether for reaction with sulfhydryl-containing targets.

In an alternate embodiment, the noncentrosymmetric metal particles can themselves be complexed to a larger object which, in turn, contains linkers for coupling the object to a molecule or particle. The Au particles can be chemically derivatized with SH-X-SH where X is an alkylthiol according to methods well known in the prior art. If a silica sphere is used, its surface can be readily derivatized with amines by using a well known reaction with an aminoalkyltrichlorosilane. The silica surface can then be covalently coupled to the Au particles via a number of commercially available heterobifunctional crosslinkers (Pierce Chemical, Inc.).

In an alternate embodiment, metallic or semiconductor particles (either centrosymrnmetric or non-centrosymmetric) can be coupled to an SHG-active particle (such as oxazole, a stryrl dye, or some other molecule or particle). These resonantly enhancing particles are well known in the prior art to strongly increase the intensity of nonlinear light scattered from a nearby nonlinear active moiety. For example, gold nanoparticles have been used to strongly enhance the SH-activity of a styryl dye [14]. Because these resonantly enhancing particles are not themselves generating the nonlinear light, they can be centrosymmetric or non-centrosymmetric. They must be close enough to the SH-active moiety to create the resonant enhancement effect, which occurs

through a dipole-dipole interaction; the distance between the two species is typically on the order of angstroms to nanometers. The general resonance enhancement effect on nonlinear optical phenomena is discussed in the context of roughened silver surfaces in references 15 and 16. The resonantly enhancing particles are available commercially with a variety of surface chemistries amenable to coupling to an SH-active molecule such as oxazole (succinimidyl ester, maleimide, etc. offered by Molecules Probes, Eugene, Oreg.). Or the particle-nonlinear-active moiety complex can be constructed according to a number of schemes available in the prior art.

In an alternate embodiment, groups or chains of the metallic particles bound together via linking molecules can be used as labels with additional functional linkers to the targets. For example, Au particles can first be sparsely derivatized with linkers for a variety of targets according to the method of the preferred embodiment. The remaining underivatized surface area on the particles can then be used to chemically couple the particles together via prior art chemistry involving dimercapto-alkyl chains

In an alternate embodiment, the particles can be centrosymmetric (eg., a sphere or a cube shape) if their size (i.e., diameter or edge length, respectively) is larger than about 10% of the wavelength of the fundamental light. For instance, a spherical particle of 80 nm diameter is expected to produce a nonlinear response when illuminated with 800 nm wavelength fundamental light.

Proteins as Nonlinear-Active Labels

Some proteins are strongly SH-active and can be used as fusion-protein labels for in-situ or in-vivo studies. For example, the gene encoding a protein of interest X can be fused at the N-terminal or C-terminal to a gene encoding one of the SH-active proteins. These include, but are not limited to, green fluorescence protein (GFP) and bacteriorhodopsin. Detailed procedures in the prior art exist for creating the fusion of such proteins to another protein of interest (references 12 and 13). For example, GFP (an intrinsically fluorescent protein) can be fused to many other proteins in order to render those proteins fluorescent GFP has been used in this way in prior art to monitor gene expression and cellular location of the GFP-X construct via fluorescence detection.

### DESCRIPTION OF THE DRAWINGS

FIG. 1:

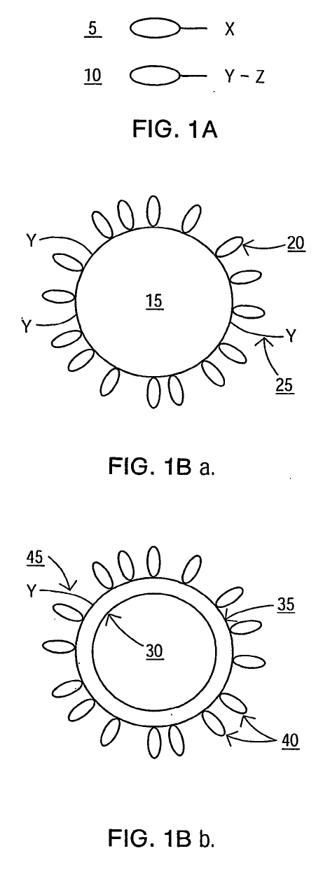

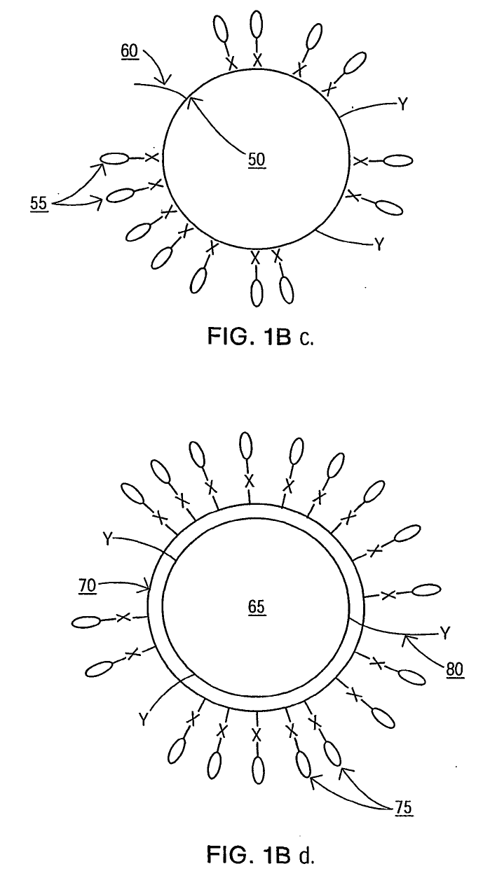

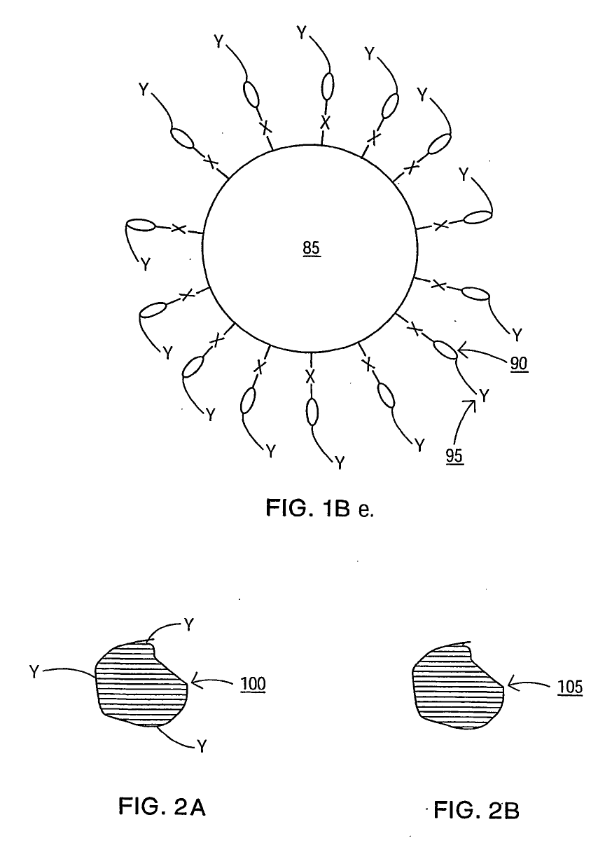

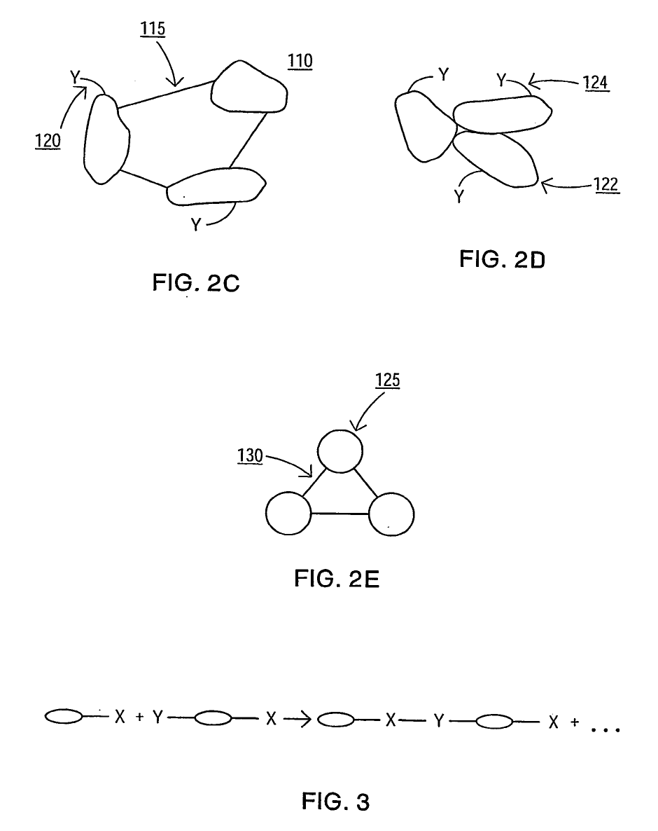

A. Organic Molecules

In the first drawing, (5) represents an organic, nonlinear-active moiety or molecule and (X) denotes the functional group which is reactive towards a target, a linker, a surface layer or a solid object.

In the second drawing, (10) represents an organic, nonlinear-active moiety or molecule and (Y-Z) denotes a linker molecule where (Y) is the group which bonds the linker to the nonlinear active moiety or molecule and Z is the functional group which is reactive towards a target, a linker, a surface layer or a solid object.

B. Derivatized Particle Labels

In drawing (a), (15) represents a solid object used as a ‘scaffold’ whose surface area is used to attach linkers (Y) and nonlinear-active moieties, molecules or particles (X). In this case, the linker Y is directly attached to the surface groups of the solid object and the nonlinear active components (20) are non-covalently adsorbed to the surface of the solid object. Functional group (Y) on the linker (25) is reactive towards a target, another linker, or a solid object.

In drawing (b), (30) represents a solid object used as a ‘scaffold’ whose surface area is used to attach linkers and nonlinear-active moieties, molecules or particles (40), (35) is a surface derivatized layer and (45) is a linker group. Functional group (Y) on the linker is reactive towards a target, another linker, or another solid object.

In drawing (c), (50) represents a solid object used as a ‘scaffold’ whose surface area is used to attach linkers and nonlinear-active moieties, molecules or particles, (55) are the covalently attached nonlinear-active moieties, molecules or particles and (60) represents the linker. In this case, (X) is a functional group on the nonlinear-active component which covalently reacts with a surface group on the solid object and (Y) is the functional group on the linker which is reactive towards a target, another linker or another solid object.

In drawing (d), (65) represents a solid object, (70) a surface layer which derivatizes the surface of the solid object and presents a functional group to the nonlinear-active components (75). (80) represents a linker which is directly attached to the surface of the solid object while (X) denotes the functional group on the nonlinear-active component which is reactive towards the functional group of the surface derivatization layer.

In drawing (e), (85) represents a solid object, (90) represents a nonlinear-active component, (X) denotes the functional group of the nonlinear-active component which allows the latter to be covalently linked to the surface of the solid object, and (Y) denotes the functional group of a linker (95) which is also part of the non-linear active component. (Y) is reactive towards a target, another linker or another solid object.

FIG. 2: Metallic and Semiconductor Particles

In drawing (A), a non-centrosymmetric metallic or semiconductor particle (100) is derivatized with linkers containing functional end-groups (Y). The functional groups (Y) is reactive towards a target, another linker or another solid object. The shape of the metallic particle (1) is drawn to emphasize its non-centrosymmetric nature.

In drawing (B), a non-centrosymmetric metallic or semiconductor particle (105) is capable of directly attaching to a target without the need for a linker.

In drawing (C) non-centrosymmetric particles (110) are attached together covalently via linkers (115) to create a composite particle which is overall non-centrosymmetric. Another linker (120) is used to covalently link the composite to a target or solid object via an end-functional group (Y).

In drawing (D), non-centrosymmetric particles (122) are adsorbed or aggregated together to create a composite particle which is overall non-centrosymmetric. Linkers (124) containing an end-functional group (Y) are used to link the composite to a target or solid object.

In drawing (E), centrosymmetric particles (125) are connected to each other via linkers (130) to create a composite particle which is overall non-centrosymmetric. As drawn, the centrosymmetric particles are covalently linked to each other via linkers, but they can also be adsorbed or aggregated to each other to create the composite. The spherical shape of the particles is intended to emphasize their centrosymmetry.

FIG. 3: Linear chains of nonlinear active moieties where X indicates a chemically reactive group on a first nonlinear active moiety capable of bonding to Y on a second nonlinear active moiety. The moieties can be assembled into a chain of desired length.

### REFERENCES

[1] “Syntheses and Photophysical Properties of some 5(2)-Aryl-2(5)-(4-pyridyl)oxazoles and Related Oxadizoles and Furans”, J. H. Hall et al., J. Heterocyclic Chem., 29, (1992) 1245.

[2] “Femtosecond decay-time measurement of electron-plasma oscillation in nanolithographically designed silver particles”, B. Lamprecht et al., Appl. Physics B, 64 (1997) 269-272.

[3] “Enormous Hyper-Rayleigh Scattering from Nanocrystalline Gold Particle Suspensions”, F. W. Vance et al., J. Phys. Chem. B, 102 (1998) 10091.

[4] “Kinetically controlled growth and shape formation mechanism of platinum nanoparticles”, J. M. Petroski et al., J. Phys. Chem. B, 102 (1998) 3316.

[5] “SHG studies of plasmon dephasing in nanoparticles”, B. Lamprecht et al., Appl. Phys. B (1997).

[6] “A truncated icosahedral structure observed in gold nanoparticles”, Surface Science, v. 447 (2000), 73.

[7] “Optical properties of a family of Au-nanoparticle-containing alumina membranes in which the nanoparticle shape is varied from needle-like (prolate) to spheroid, to pancake-like (oblate)”, Thin Solid Films, v. 303, (1997), 84-8.

[8] “Thiol-derivatized nanocrystalline arrays of gold, silver, and platinum”, J. Phys. Chem. B., v. 101, (1997) 9876-80.

[9] “Gigantic optical non-linearities from nanoparticle-enhanced molecular probes with potential for selectively imaging the structure and physiology of nanometric regions in cellular regions”, Bioimaging, v. 4 (1996), 215.

[10] “Second Harmonic Spectroscopy: Detection and Orientation of Molecules at a Biomembrane Interface”, J. S. Salafsky and K. B. Eisenthal, Chemical Physics Letters 2000, 319, 435-439.

[11] “Protein Adsorption at Interfaces Detected by Second Harmonic Generation”, J. S. Salafsky and K. B. Eisenthal, Journal of Physical Chemistry B, 2000, 104, 7752-7755.

[12] Protein Expression, A Practical Approach, Ed. S. J. Higgins and B. D. Hames, Oxford University Press, 1999.

[13] Cubitt, A. B. et al., Trends Biochem. Sci., 20 (1995), 448.

U.S. Pat. No. 5,521,289, “Small organometallic probes”, R. D. Powell.

U.S. Pat. No. 5,728,590, “Small organometallic probes”, R. D. Powell.

[14] “Nonlinear optical measurement of membrane potential around single molecules at selected cellular sites”, G. Peleg, A. Lewis, M. Linial, L. M. Loew, Proc. Natl. Acad. Sci. V. 96, 1999, 6700-6704.

[15] G. Boyd, T. Rasing, R. Leite, Y. Shen (1984) Phys. Rev. B 30, 519-526.

[16] C. Chen, T. F. Heinz, D. Richard, Y. Shen (1981) Phys. Rev. Lett. 46, 1010-1012.

What is claimed is:

1. Nonlinear-active labels, said labels capable of being attached to a target for the purpose of studying said target using a surface-selective nonlinear optical technique.

2. The label according to claim 1, wherein the label includes a moiety selected from the group consisting of:
Oxazole or oxadizole molecules
5-aryl-2-(4-pyridyl)oxazole
2-aryl-5-(4-pyridyl)oxazole
2-(4-pyridyl)cycloalkano[d]oxazoles
Merocyanines
Stilbenes
Indodicarbocyanines
Hemicyanines
Stilbazims
Azo dyes
Cyanines
Stryryl-based dyes
Methylene blue
Diaminobenzene compounds
Polyenes
Diazostilbenes
Tricyanovinyl aniline
Tricyanovinyl azo
Melamines
Phenothiazine-stilbazole
Polyimides
Sulphonyl-substituted azobenzenes
Indandione-1,3-pyidinium betaine
Fluoresceins
Benzooxazoles
Perylenes
Polymethacrylates
Oxonols
Thiophenes
Bithiophenes

3. The label of claim 1, which includes an oxazole moiety based on a 1,3-oxazole.

4. The label of claim 2, wherein the oxazole moiety is a quaternary salt.

5. The label of claim 2, wherein the oxazole moiety is 2-(4-N-methylpyridinium)-4,5-dihydronaphtho [2,1-d]-1,3-oxazole p-toluenesulfonate.

6. The label of claim 2, wherein the oxazole is 2-(4-N-methylpyridinium)-4,5-dihydro6-methoxynaphtho[2,1-d]-1,3-oxazole p-toluenesulfonate.

7. The label of claim 2 wherein the oxazole moiety is a 5(2)-Aryl-2(5)-(4-pyridyl)oxazole

8. The label of claim 2 wherein the oxazole moiety is a 2,5-Diaryl-1,3-oxazole

9. The label of claim 1 wherein the label is: 1-(3-(succinimidyloxycarbonyl) benzyl)-4-(5-(4-methoxyphenyl) oxazol-2-yl)pyridinium bromide

10. The label of claim of 1 wherein the label is: 1-(2,3-epoxypropyl)-4-(5-(4-methoxyphenyl)oxazol-2-yl) pyridinium trifluoromethanesulfonate (PyMPO epoxide).

11. The label of claim 1 wherein the nonlinear-active label includes a non-centrosymmetric metallic or semiconductor particle.

12. The label of claim 1, wherein the nonlinear-active label includes a centrosymmetric metallic or semiconductor particle with a size of greater than or equal to 10% of the wavelength of the fundamental light.

13. The label of claim 1 which comprises bacteriorhodoposin or the green fluorescent protein.

14. The label of claim 1 including a genetically engineered mutant of bacteriorhodopsin or the green fluorescent protein.

15. The label of claim 1 wherein a target object is first derivatized with a linker and then coupled to the label.

16. The label according to claims 2-14 wherein the nonlinear active moieties, particles or proteins are coupled to a linker molecule.

17. A non-linear active label capable of attachment to a target for the purpose of studying the target using a surface-selective nonlinear optical technique, wherein said label comprises a solid object to be used as a scaffold which provides a surface area onto which is attached at least one nonlinear-active component.

18. The label of claim 17 wherein the nonlinear-active components include one or more of the following: a moiety from claims 2-10, a particle from claims 11 and 12 or a protein from claims 13 and 14.

19. The label of claim 18 wherein the components include a linker molecule for attachment to the solid surface.

20. The label of claim 17 wherein a linker molecule is attached directly to the surface of the solid object, said linker allowing the coupling of the label to the target.

21. The label of claim 17 wherein a linker molecule is attached to the solid surface via a derivatized surface layer, said layer being attached to the surface, said linker allowing coupling of the layer to the target.

22. The label of claim 18 wherein the components include a linker molecule for attachment to a target.

23. The label of claim 17 wherein the solid object is derivatized with a surface layer.

24. The claims of 17 and 18 wherein the nonlinear active component is attached to the surface layer.

25. The claims of 21 and 23 wherein the surface layer is a self-assembled monolayer

26. The label of claim 25 wherein the surface layer is in the chemical family of silane compounds.

27. The label of claim 17, wherein the solid object is a particle, cluster, colloidal particle, nanocrystal or nanoparticle of size scale ranging from nanometers to microns.

28. The label of claim 17, wherein the solid object is composed of a polymer, latex, polystyrene, silica, glass or silicon.

29. The label of claims 19-21 wherein the linker is longer than 8 carbon-atom lengths.

30. The claims of 11-12 wherein the particle comprises Au, Ag, Pt, CdS, CdSe, TiO₂, GaAs, InP, GaP.

31. The claims of 11-12 wherein the particles are also complexed to or attached to any of the moieties, particles or proteins in claims 2-14.

32. The label of claim 19 wherein the nonlinear-active particle is attached to the solid surface using functionalized alkylthiols.

33. A non-linear label for use in detection of a target at an interface using a nonlinear optical technique, said label comprising a metallic or semiconductor particle.

34. The label of claim 33, wherein the particle is non-centrosymmetric.

35. The label of claim 33, wherein the particle is centrosymmetric and has a size of greater than or equal to 10% of the wavelength of the fundamental light used in said nonlinear optical technique.

36. The label of claim 33, wherein the particle is chemically derivatized with a linker molecule, said linker being reactive with a target object.

37. The label of claim 33, wherein the particle is chemisorbed or physisorbed to the target.

38. The label of claim 33, wherein the particle is composed of Au, Ag, Pt, CdS, CdSe, TiO₂, GaAs, InP, GaP.

39. The label of claim 33, wherein the metallic particle is derivatized with functionalized alkylthiols.

40. The lable of claim 32 and 39, wherein the alkylthiols are functionalized with groups that are reactive toward amine, sulfhydryl, carboxylic, aldehyde, ketone, vicinal diol, glutamine, oligosaccharide, guadinium, NHS ester, methyl ester, hydroxyl, azido-methylcoumarin, Sulfo-NHS ester, maleimide, iodoacetyl, vinyl sulfone, —CH bonds, carbodiimide-activated carboxyl, biotin, streptavidin, and phosphatidylcholine moieties.

41. The label of claim 33, wherein the label consists of two or more particles in a composite, said particles being aggregated, adsorbed to each other, or attached to each other via covalent, noncovalent or electrostatic means, said composite being overall non-centrosymmetric.

42. A nonlinear active label for use in labeling a target and detecting said target using a surface-selective nonlinear optical technique, wherein said label comprises a polymeric molecule composed of two or more nonlinear-active moieties in which the moieties are coupled to each other in a linear head-to-tail fashion, and in which the orientation of each moiety in the chain is in the same direction.

43. The label of claim 42 wherein the chains are composed of nonlinear-active molecules.

44. The label of claim 42 wherein the chains are composed of any of the moieties, particles or proteins in claims 2-14.

45. A non-linear active label comprising a nonlinear-active protein.

46. The label of claim 45 wherein the target is a protein and the nonlinear-active label is a fusion protein.

47. The label of claim 45 wherein said target is a protein and the nonlinear-active label is fused to said target via co-expression of the genes encoding both proteins.

48. The label of 46 wherein the nonlinear-active protein is either bacteriorhodoposin or the green fluorescent protein.

49. The label of claim 46 wherein the nonlinear-active protein is a genetically engineered mutant of either bacteriorhodopsin or the green fluorescent protein.

50. A method for detecting a target at an interface of interest, said method comprising attaching a nonlinear active label according to any of claims 1-49 to said target and measuring said target at said interface using a surface-selective nonlinear optical technique.

51. The method of claim 50 wherein the interface is an air-water interface, a glass water interface, a solid-water interface or a solid-air interface, a vapor-liquid interface, a solid-solid-interface, or a liquid-liquid interface, cellular interface, membrane interface.

52. The method of claim 50 or 51 wherein the target is a protein, oligosaccharide, peptide, nucleic acid, liposome, small molecule, oligonucleotide, liposome, or biological cell, oligosaccharide, antibody, antigen, peptide, virus, receptor, drug, enzyme, ligand, carbohydrate.

53. The method of any of claims 50-52 wherein the nonlinear active technique is second harmonic generation, sum frequency generation or difference frequency generation.

54. The method of any of claims 50-53 wherein the targets are derivatized with biotin molecules and the nonlinear active labels are derivatized with streptavidin.

55. The labels according to any of claims 1-49 which are derivatized with biotin.

56. The method of any of claims 50-55 wherein said label is according to any of claims 19-22, 33 and 36 wherein the linkers include one or both of the following: streptavidin, biotin.

57. The labels according to any of claims 5,16,19-22,29,36 and 54 wherein the linkers are functionalized with groups that are reactive toward amine, sulfhydryl, carboxylic, aldehyde, ketone, vicinal diol, glutamine, oligosaccharide, guanidium NHS ester, methyl ester, hydroxl, azido-methylcoumarin, Sulfo-NHS ester, maleimide, iodoacetyl, vinyl sulfone, —CH bonds, carbodiimide-activated carboxyl, biotin, streptavidin, and phosphatidylcholine moieties.

58. The label according to any of claims of 15,16,19-22,29,36 and 54 wherein the linkers contain a functional group which is reactive towards a target, a second linker, a surface layer or a solid object.

59. The label according to claim 17 wherein the solid object contains a surface functional group which is reactive towards a target, a linker, a surface layer or other solid objects.

60. The label according to any of claims 2-14 or 33 wherein the nonlinear active moiety, molecule or particle contains a functional group which is reactive towards a target, a linker, a surface layer or a solid object

61. The label according to claims 17, 27 or 28 wherein the solid object is spherical in shape, bead-like or quasi-spherical.

62. The label according to claim 1, comprised of two or more distinct kinds of species comprising a single label, wherein the effect of the first kind of said species is to resonantly enhance the nonlinear activity of the second kind of said species.

63. The label according to claim 62, wherein the first kind of species are metallic or semiconductor particles and are used to create the resonance enhancement effect.

64. The label according to claim 62, wherein the metallic or semiconductor particles are centrosymmetric or non-centrosymmetric.

65. The label according to claim 62, wherein the second kind of species is any optically nonlinear active moiety, molecule or particle.

66. The label according to claim 62, wherein the two kinds of species are chemically bonded, attached, or linked to each other.

67. The label according to claim 66, wherein the average distance between the two kinds of species is of order angstroms or nanometers.
